(19) United States
(12) Patent Application Publication
(10) Pub. No.: US 2024/0122727 A1
(43) Pub. Date: Apr. 18, 2024

(54) ACETABULAR CUP EXTRACTOR

(71) Applicant: Shukla Medical, St. Petersburg, FL (US)

(72) Inventor: Zachary Robert Sweitzer, Keyport, NJ (US)

(73) Assignee: Shukla Medical, St. Petersburg, FL (US)

(21) Appl. No.: 18/459,904

(22) Filed: Sep. 1, 2023

Related U.S. Application Data
(63) Continuation-in-part of application No. 29/856,578, filed on Oct. 14, 2022.

(51) Int. Cl. A61F 2/46

(52) U.S. Cl. CPC A61F 2/4609; A61F 2/4603; A61F 2002/4619

(57) ABSTRACT

An orthopedic instrument in the form of an acetabular cup extractor for reliably and efficiently extracting an acetabular cup implant from an acetabulum. The acetabular cup extractor includes a handle assembly and a quick connect coupling extending from the handle assembly. The acetabular cup extractor further includes a blade having a first quick connect for connecting with the quick connect coupling and a second quick connect. In addition, the acetabular cup extractor includes a guide configured to be received within an acetabular cup, the guide including a quick connect coupling for connecting with the second quick connect of the blade.

### CROSS-REFERENCE TO RELATED APPLICATIONS

This application is a continuation-in-part of U.S. patent application Ser. No. 29/856,578, filed Oct. 14, 2022, the disclosure of which is incorporated herein in its entirety for all purposes.

### BACKGROUND

The exemplary embodiments of present invention relate generally to an orthopedic instrument and, more specifically, to an acetabular cup extractor.

In performing hip arthoplasty surgery, an acetabular cup implant is installed in the hip socket or acetabulum. When installing the acetabular cup implant into the acetabulum, bone is removed from the acetabulum to receive the acetabular cup implant and the acetabular cup implant is attached, e.g., with cement, bone screws or press-fitted. The acetabular cup implant may have a porous, typically metal coating shell which surrounds a liner adapted to receive the ball-like head of a hip stem implant. Bone gradually grows onto and/or into the porous metal shell to permanently affix the acetabular cup implant to the acetabulum.

Prosthetic hip joints, like their natural counterparts, experience wear over time. When a prosthetic hip joint becomes worn or damaged to a point necessitating maintenance or replacement, the acetabular cup implant may have to be replaced. In order to replace the acetabular cup implant, an extractor is used to extract the existing acetabular cup implant from the acetabulum.

Among the disadvantages of conventional extractors is that the blades are either permanently attached to the handle or attached with a threaded fastener which actions may prolong surgery time. Conventional extractors have blades with flat cutting edges which require substantial force to plunge the blades into the bone surrounding the acetabular cup implant being extracted. Handles of typical extractors also are not ergonomically shaped which render them uncomfortable to the user because of the substantial force that must be applied to the handle during use. Conventional extractors also typically have a single impact surface to dislodge the blade in the event it gets stuck in the bone, which may not be optimally positioned for striking.

In addition, in conventional extraction systems having a spherical head that caps an implant the spherical head can easily separate from the acetabular cup liner when the handle is used to drive the blade into bone, e.g., by being struck by a hammer. Such separation of the spherical head detrimentally impacts the user's ability to control the point of insertion of the blade into bone. As a consequence, the blade may make an irregular cut of the surrounding bone whereby more bone than necessary may be cut in order to extract the acetabular cup from the acetabulum. In revision surgery to remove existing implants, as little bone as possible should be removed from around the acetabular cup in order to preserve the integrity of the surrounding bone.

### BRIEF SUMMARY OF THE DISCLOSURE

In accordance with an exemplary embodiment there is provided an acetabular cup extractor comprising a handle assembly, a quick connect coupling extending from the handle assembly, a blade having a first quick connect for connecting with the quick connect coupling and a second quick connect, and a guide configured to be received within an acetabular cup, the guide including a quick connect coupling for connecting with the second quick connect of the blade.

According to an aspect, the handle assembly comprises a first handle having a first shaft portion extending from the quick connect coupling, and a second shaft portion extending from the first shaft portion and having a longitudinal axis laterally offset from a longitudinal axis of the first shaft portion. According to another aspect, the second shaft portion is offset from the longitudinal axis of the first shaft portion a distance substantially equal to a distance a tip of the blade is offset from a longitudinal axis of the first quick connect. According to another aspect, the blade has a tip substantially in-line with the longitudinal axis of the second shaft portion. According to another aspect, the first handle further comprises a strike plate at its proximal end and positioned directly above the blade.

According to an aspect, the handle assembly further comprises a second handle having a longitudinal axis substantially transverse to a longitudinal axis of the first handle. According to another aspect, the second handle has a T-handle at a first end of the second handle, and an enlarged gripping portion about a second end of the second handle opposite the first end. According to another aspect, the second handle includes a striking face. According to another aspect, the T-handle includes a first striking face and a second striking face opposite the first striking face. According to another aspect, the enlarged gripping portion includes a first striking face and a second striking face opposite the first striking face.

According to an aspect, the first quick connect extends coaxially with the second quick connect. According to another aspect, the first quick connect extends from the blade in a first direction and the second quick connect extends from the blade in a second direction opposite the first direction. According to another aspect, the blade includes a spear-point blade. According to another aspect, the blade is curved in both its longitudinal direction and in a widthwise extent transverse to the longitudinal direction. According to another aspect, the blade has an arc length of about 80 to 140 degrees.

In accordance with another exemplary embodiment there is provided an acetabular cup extractor comprising a blade including a first quick connect and a second quick connect, a quick connect coupling for connecting with the first quick connect of the blade, a handle assembly, and a guide configured to be received within an acetabular cup, the guide including a quick connect coupling for connecting with the second quick connect of the blade. The blade further includes a spear-point blade having a tip that is substantially in-line with the longitudinal axis of the second shaft portion, and wherein the blade is curved in both its longitudinal direction and in a widthwise extent transverse to the longitudinal direction. The handle assembly includes a first handle and a second handle having a longitudinal axis substantially transverse to a longitudinal axis of the first handle. The first handle has a first shaft portion extending from the from the quick connect coupling, a second shaft portion extending from the first shaft portion and having a longitudinal axis laterally offset from a longitudinal axis of the first shaft portion, and a strike plate at its proximal end. The second handle portion has a T-handle at a first end of the second handle, and an enlarged gripping portion about a second end of the second handle opposite the first end, wherein the enlarged gripping portion includes a planar striking face.

Other features and advantages of the subject disclosure will be apparent from the following more detail description of the exemplary embodiments.

### BRIEF DESCRIPTION OF THE SEVERAL VIEWS OF THE DRAWINGS

The foregoing summary, as well as the following detailed description of the exemplary embodiments of the subject disclosure, will be better understood when read in conjunction with the appended drawings. For the purpose of illustrating the present disclosure, there are shown in the drawings exemplary embodiments. It should be understood, however, that the subject application is not limited to the precise arrangements and instrumentalities shown.

FIG. 1 is a left-side elevational view of an acetabular cup extractor in accordance with an exemplary embodiment of the subject disclosure;

FIG. 2 is a perspective view of a handle assembly of the acetabular cup extractor of FIG. 1;

FIG. 3 is a partial cross-sectional view of the acetabular cup extractor of FIG. 1;

FIG. 4 is a perspective view of a blade of the acetabular cup extractor of FIG. 1;

FIG. 5 is a side elevational view of the blade of FIG. 5;

FIG. 6 is a perspective view of a guide of the acetabular cup extractor of FIG. 1;

FIG. 7 is a top plan view of the guide of FIG. 6;

FIG. 8 is a side elevational view of the blade of FIG. 4 shown connected to the guide of FIG. 6;

FIG. 9 is a side elevational view of a second exemplary embodiment of a blade shown connected to a second exemplary embodiment of a guide of an acetabular cup extractor in accordance with another exemplary embodiment of the subject disclosure, wherein the guide is shown in contact with an acetabular cup implant;

FIG. 10 is a bottom perspective view of the blade and guide of FIG. 9; and

FIG. 11 is a cross-sectional view blade and guide of FIG. 9 taken along line 11-11 thereof.

### DETAILED DESCRIPTION OF THE DISCLOSURE

Reference will now be made in detail to the various exemplary embodiments of the subject disclosure illustrated in the accompanying drawings. Wherever possible, the same or like reference numbers will be used throughout the drawings to refer to the same or like features. It should be noted that the drawings are in simplified form and are not drawn to precise scale. Certain terminology is used in the following description for convenience only and is not limiting. Directional terms such as top, bottom, left, right, above, below and diagonal, are used with respect to the accompanying drawings. The term “distal” shall mean away from the center of a body. The term “proximal” shall mean closer towards the center of a body and/or away from the “distal” end. The words “inwardly” and “outwardly” refer to directions toward and away from, respectively, the geometric center of the identified element and designated parts thereof. Such directional terms used in conjunction with the following description of the drawings should not be construed to limit the scope of the subject application in any manner not explicitly set forth. Additionally, the term “a,” as used in the specification, means “at least one.” The terminology includes the words above specifically mentioned, derivatives thereof, and words of similar import.

“About” as used herein when referring to a measurable value such as an amount, a temporal duration, and the like, is meant to encompass variations of ±20%, ±10%, ±5%, ±1%, or ±0.1% from the specified value, as such variations are appropriate.

“Substantially” as used herein shall mean considerable in extent, largely but not wholly that which is specified, or an appropriate variation therefrom as is acceptable within the field of art. “Exemplary” as used herein shall mean serving as an example.

Throughout the subject application, various aspects thereof can be presented in a range format. It should be understood that the description in range format is merely for convenience and brevity and should not be construed as an inflexible limitation on the scope of the subject disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

Furthermore, the described features, advantages and characteristics of the exemplary embodiments of the subject disclosure may be combined in any suitable manner in one or more embodiments. One skilled in the relevant art will recognize, in light of the description herein, that the subject disclosure can be practiced without one or more of the specific features or advantages of a particular exemplary embodiment. In other instances, additional features and advantages may be recognized in certain embodiments that may not be present in all exemplary embodiments of the present disclosure.

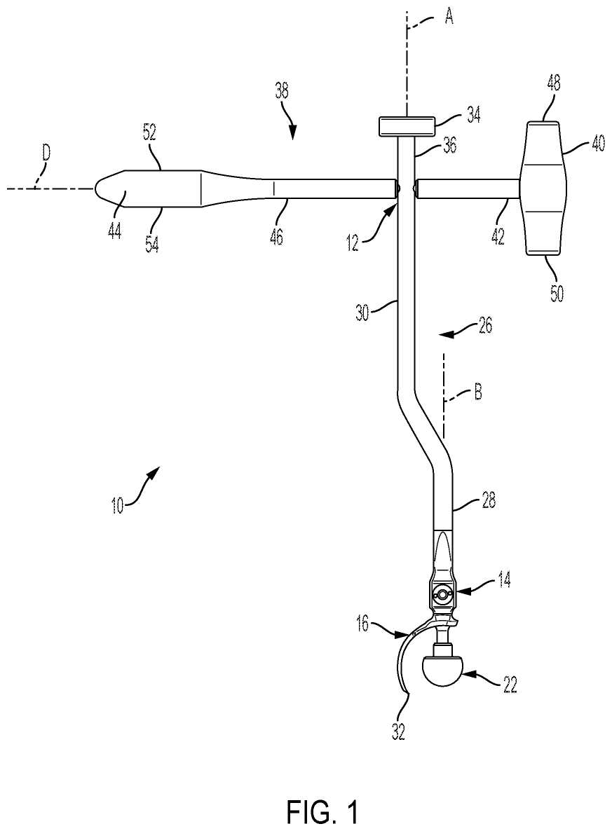

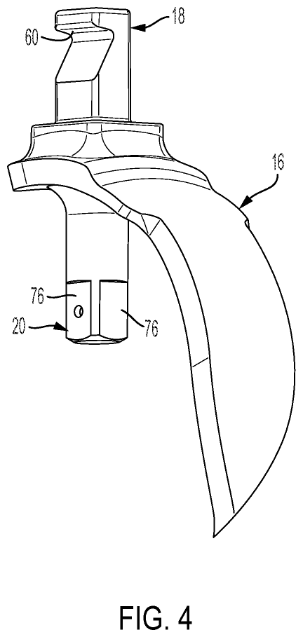

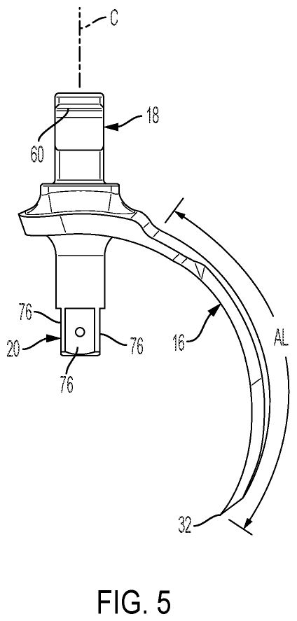

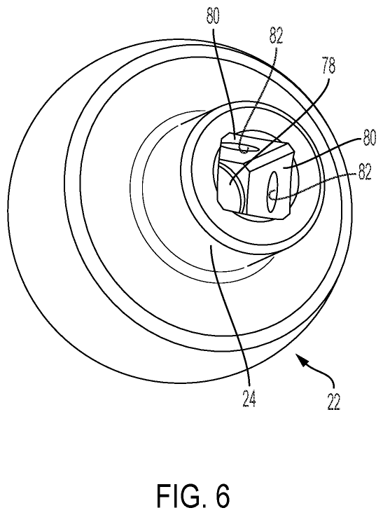

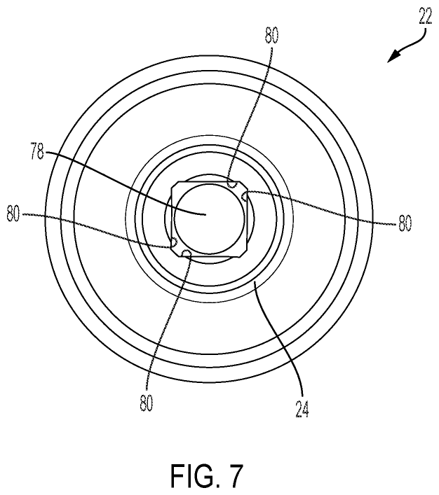

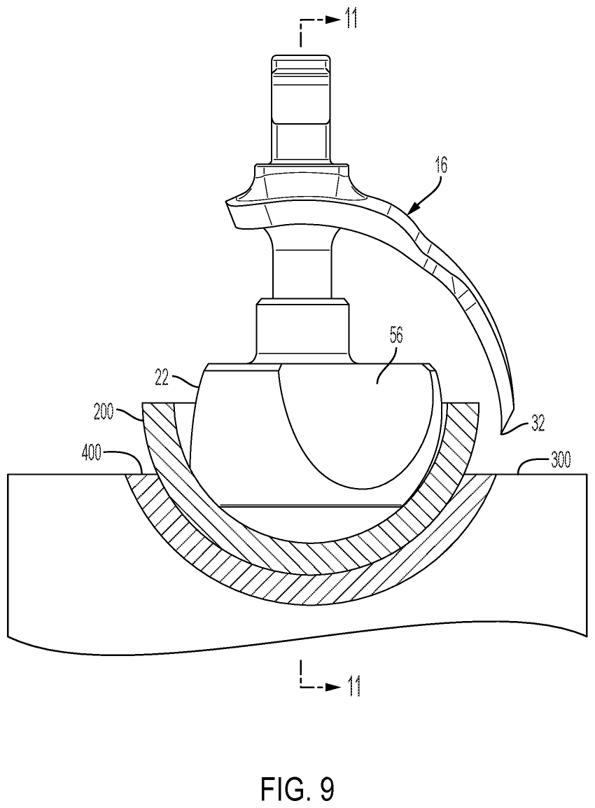

Referring now to the drawings, FIG. 1 illustrates an exemplary embodiment of an acetabular cup extractor 10 in accordance with the subject disclosure. The acetabular cup extractor 10 includes a handle assembly 12 and a quick connect coupling 14 extending from the handle assembly. As best shown in FIGS. 4 and 5, the acetabular cup extractor further includes a blade 16 having a first quick connect 18 for connecting with the quick connect coupling 14 and a second quick connect 20. In addition, the acetabular cup extractor includes a guide 22 configured to be received within an acetabular cup implant liner 200 (FIG. 9), the guide including a quick connect coupling 24 (FIGS. 6 and 7) for connecting with the second quick connect 20 of the blade 16.

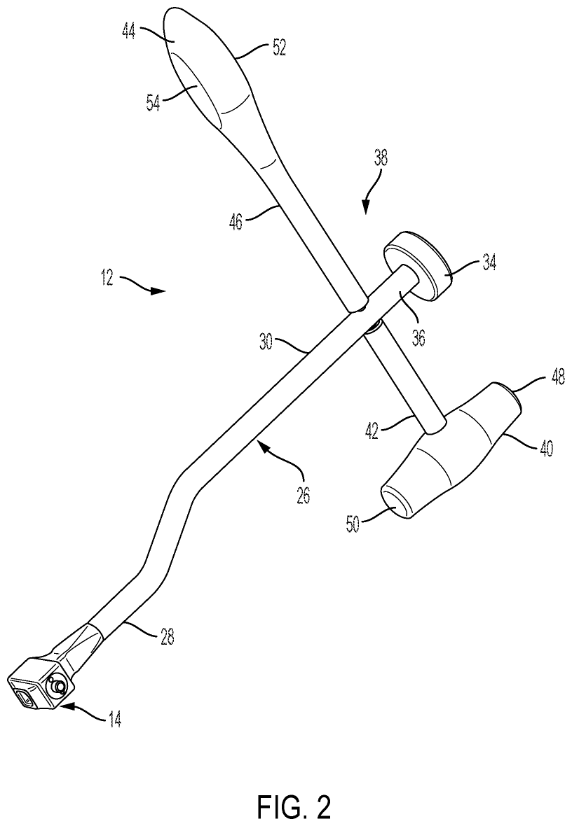

As shown in FIGS. 1 and 2, the handle assembly 12 includes a first handle 26 having a first shaft portion 28 extending from the quick connect coupling 14, and a second shaft portion 30 extending from the first shaft portion and having a longitudinal axis “A” laterally offset from a longitudinal axis “B” of the first shaft portion. According to an exemplary embodiment, the second shaft portion 30 is offset from the longitudinal axis “B” of the first shaft portion a distance substantially equal to a distance a tip 32 of the blade 16 is offset from a longitudinal axis “C” (FIG. 5) of the first quick connect 18 of the blade. That is, as shown in FIG. 1, the blade 16 has a tip 32 substantially in-line with the longitudinal axis “A” of the second shaft portion 30. In addition, the first handle 26 further comprises a strike plate 34 at its proximal end 36 and positioned directly above the blade.

The handle assembly 12 further comprises a second handle 38 having a longitudinal axis “D” substantially transverse to a longitudinal axis, i.e., either axis “A” or axis “B”, of the first handle 26. According to an exemplary embodiment, the second handle 38 has a T-handle 40 at a first end 42 of the second handle, and an enlarged gripping portion 44 about a second end 46 of the second handle opposite the first end. The second handle includes a striking face. According to an exemplary embodiment, the T-handle 40 includes a first striking face 48 and a second striking face 50 opposite the first striking face, and the enlarged gripping portion 44 includes a first striking face 52 and a second striking face 54 opposite the first striking face. The second striking face 50 and the first striking face 52 may be struck by a striking tool such as a hammer, mallet or the like, in lieu of or addition to the proximal face of the of the strike plate 34 when driving the blade 16 into bone 300 surrounding an acetabular implant 400 (FIG. 9). Conversely, the first striking face 48 and the second striking face 54 may be struck by a striking tool in order to dislodge the blade 16 and acetabular cup 200 from bone 300.

The first handle 26 can have an overall length of about 6 to about 30 inches, preferably about 8 to about 24 inches, and more preferably about 10 to about 18 inches, but can be more or less than the foregoing ranges. In an exemplary embodiment, the first handle 26 has an overall length greater than an overall longitudinal length of the second handle 38. In another exemplary embodiment, the first handle 26 has an overall length substantially the same as an overall longitudinal length of the second handle 38.

The masses and locations of the T-handle 40 and the enlarged gripping portion 44 are preferably such that substantial balance is achieved when the handle assembly 12 is held in an upright position. In an exemplary embodiment, the handle assembly has a mass of about 1 to 2 lbs., and preferably about 1.5 lbs., but can be more than 2 lbs. or less than 1 lb. A mass of the second handle 38 about the first lateral side of the first handle 26 is substantially the same as a mass of the second handle about the second lateral side of the first handle such that forces applied to the T-handle 40 produces substantially similar torque force when a force is applied to the enlarged gripping portion 44. In this way, torqueing of the handle assembly 12 is made easier when the blade 16 is inserted into bone surrounding an acetabular implant. In addition, the balance imparted by the handle assembly 12 assures firm and reliable contact between the mating surfaces of the guide 22 and the acetabular cup 200.

Referring to FIGS. 4 and 5, according to an exemplary embodiment, the first quick connect 18 extends from the blade 16 in a first direction and the second quick connect 20 extends from the blade in a second direction opposite the first direction, wherein the first quick connect extends coaxially with the second quick connect. The blade 16 is an arcuate or curved blade so as to closely accommodate the outer shape of an acetabular cup implant to be extracted. As best shown in FIG. 4, the blade includes a spear-point blade and is curved in both its longitudinal direction and in a widthwise extent transverse to the longitudinal direction. And, as shown in FIG. 5 the blade 16 has an arc length “AL” of about 80 to 140 degrees, including 70, 75, 85, 90, 95, 100, 105, 110, 115, 120, 125, 130, 135, 145 and 150 degrees.

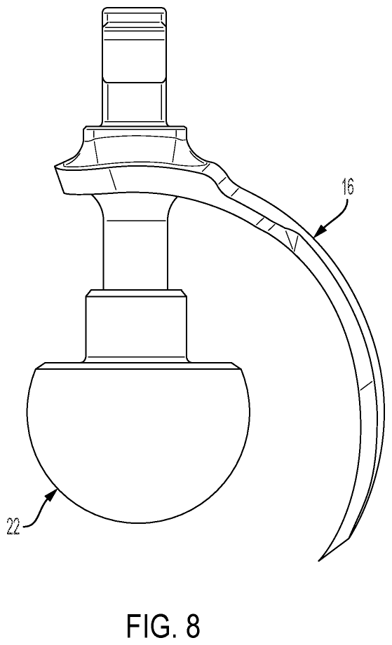

As shown in FIGS. 1, 3 and 8, according to an exemplary embodiment of the subject disclosure, the guide 22 is substantially shaped as a frustrum of a hemispherical dome.

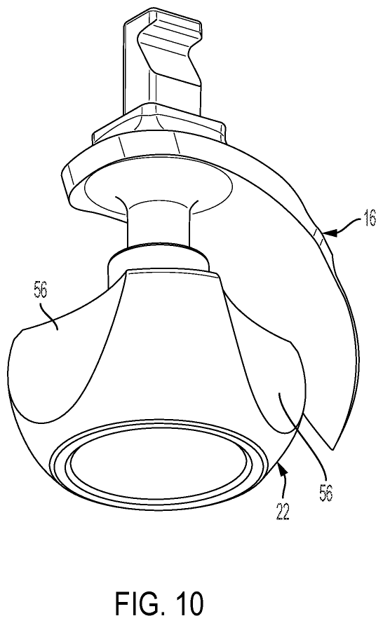

As shown in FIGS. 9-11, according to another exemplary embodiment of the subject disclosure, the guide 22 can include a plurality of spaced apart scallop-shaped recesses 56 circumscribing the guide to facilitate gripping of the guide by a user.

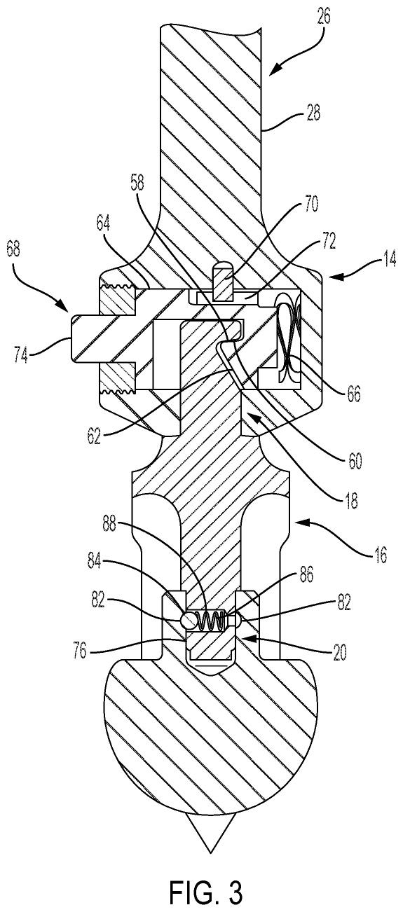

Referring to FIG. 3, the quick connect coupling 14 of the first handle 26 is positioned about a distal end of the first shaft portion 28 for connecting to the first quick connect 18. In an exemplary embodiment, the quick connect coupling 14 includes a detent 58 for engaging a cooperating detent 60 of the first quick connect 18. The quick connect coupling 14 is configured to include a ramped surface 62 (which is part of a sliding latch member 64). The quick connect coupling 14 further includes a biasing member 66 for biasing the detent 58 into engagement with the cooperating detent 60 of the first quick connect 18. The biasing member 66 can be any suitable biasing member, e.g., a coil spring, a leaf spring, an elastomer, and the like.

A release mechanism 68 enables a user to overcome the biasing force of the biasing member 66 and release the detents 58, 60 from one another when it is desired to remove the first quick connect 18 from the quick connect coupling 14. In an exemplary embodiment, the quick connect coupling 14 includes a centering member 70 for engaging a slot 72 provided in the sliding latch member 64 to maintain the orientation and securing of the sliding latch member 64 during engagement and disengagement of the detents 58, 60. The sliding latch member 64 further includes a button 74 which protrudes from a sidewall of the quick connect coupling 14 adapted to be pressed inwardly by a user when it is desired to disengage the first quick connect 18 from the quick connect coupling 14.

Engagement of the first quick connect 18 with the quick connect coupling 14 is as follows. Together, the first quick connect 18 and the quick connect coupling 14 comprise a push-to-connect assembly. That is, the first quick connect 18 is simply pushed into the quick connect coupling 14 in order to connect the first quick connect to the quick connect coupling. More particularly, as the first quick connect is pushed into the quick connect coupling, the detent 60 of the first quick connect contacts the ramped surface 62, thereby displacing the sliding latch member 64 in a first direction against the bias of the biasing member 66, thereby compressing the biasing member. Once the detent 60 has passed the ramped surface 62, the biasing member 66 expands thereby displacing the sliding latch member 64 in a second direction opposite the first direction, whereby the detent 58 passes under and interlocks with the detent 60. With the detents 58 and 60 interlocked, the first quick connect is firmly engaged with the quick connect coupling such that the first quick connect cannot be disengaged from the quick connect coupling simply by pulling on the first quick connect.

Disengagement of the first quick connect 18 from the quick connect coupling 14 is as follows. A user depresses the button 74 which displaces the sliding latch member 64 in a first direction against the bias of the biasing member 66, thereby compressing the biasing member. The user continues to depress the button 74 until the detent 58 is no longer under and interlocked with the detent 60. With the button 74 sufficiently depressed, i.e., the detent 60 is “clear” of the detent 58, the user simply withdraws the first quick connect 18 from the quick connect coupling 14 and releases the button 74, whereby the biasing member 66 returns the sliding latch member 64 to a “ready” position suitable for again receiving the first quick connect 18.

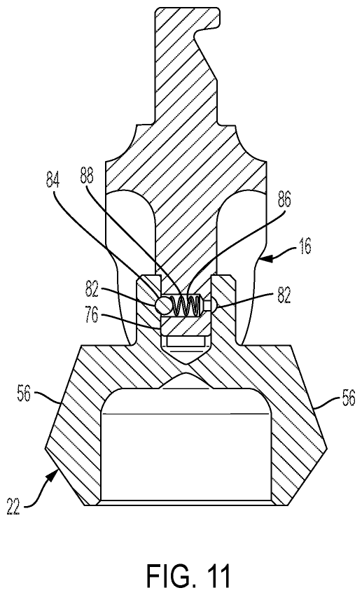

Referring to FIGS. 3-7 and 11, there is shown the constructions and interactions of the second quick connect 20 and the quick connect coupling 24 of the guide 22 according to an exemplary embodiment of the subject disclosure. Turning first to FIGS. 4 and 5, the second quick connect 20 is a male connector having a polygonal peripheral shape including a plurality of external walls 76. As illustrated, the second quick connect is square in peripheral shape, but may assume any other shape including, without limitation, triangular, pentagonal, hexagonal, or the like. Referring to FIGS. 6 and 7, the quick connect coupling 24 of the guide 22 is a female connector having a socket 78 defining a polygonal peripheral shape including a plurality of internal walls 80. As illustrated, the quick connect coupling 24 is square in peripheral shape, but may assume any other shape including, without limitation, triangular, pentagonal, hexagonal, or the like, suitable for mating with the shape of the second quick connect 20. As shown in FIG. 6, one or more of the walls 80 of socket 78 can include a hemispherical recess 82 configured to releasably receive an ball detent 84 protruding from a wall 76 of the second quick connect (see FIGS. 3 and 11). As shown in FIGS. 3 and 11, a compression spring 86 or other suitable biasing member is housed within a chamber 88 provided in the second quick connect 20 and operates to outwardly bias the ball detent 84 into one of the hemispherical recesses 82 provided in the internal walls of the socket quick connect coupling 24.

Engagement and disengagement of the second quick connect 20 and the quick connect coupling 24 of the guide 22 is as follows. In order to engage the second quick connect 20 with the quick connect coupling 24 of the guide 22, the user aligns the external walls 76 of the second quick connect 20 with the internal walls 80 of the quick connect coupling 24. The user then inserts the second quick connect into quick connect coupling until the ball detent 84, which is pushed into the chamber 88 during insertion, moves outwardly under the bias of the biasing member 86 and comes to rest in one of the hemispherical recesses 82 provided in the internal walls of the socket quick connect coupling 24. Such engagement firmly holds the guide 22 onto the second quick connect 20 to an extent sufficient to enable the user to perform an acetabular cup implant extraction procedure as described below. In order to disengage the second quick connect 20 from the quick connect coupling 24 of the guide 22, the user simply pulls on the guide with sufficient force to unseat the ball detent 84 from the hemispherical recess 82 in which it was resting and continue to pull until the second quick connect is freed from the quick connect coupling of the guide.

In operation of the acetabular cup extractor of the subject disclosure, an appropriately sized and shaped blade 16 is selected and the first quick connect thereof is attached to the quick connect coupling 14. In particular, the blade 16 shown in FIG. 9 is typically selected first as it is a starter blade, i.e., it has a shorter length than the blade 16 of FIG. 5 which is known as a finishing blade. Thereafter, an appropriately sized and shaped guide 22 is selected and attached to the second quick connect 20. The guide 22 is then seated in the acetabular cup implant liner 200 in the manner shown in FIG. 9.

The point 32 of the blade 16 is then torqued to pivot about the guide 22 to force the point to cut into bone 300 surrounding the acetabular cup implant 400 to be extracted. This movement causes the blade to cut into bone substantially the length of the blade 16. Alternatively, the user can strike the strike plate 34 of the handle assembly 12 if necessary to drive the point 32 and the lateral cutting edges of the blade into the bone. The user then extracts the blade 16 from the bone. If the blade 16 is stuck in the bone, the user can strike the first striking face 48 of the T-handle 40 or the second striking face 54 of the enlarged gripping portion 44 to free the stuck blade from the bone. Once the blade 16 is extracted from the bone, the user rotates the handle assembly 12 a desired angular distance and again places the point 32 of the blade 16 into contact with bone surrounding the acetabular cup implant to be extracted. The user then torques the handle or strikes the strike plate 34, thereby again driving the blade 16 into the bone. The user then extracts the blade from the bone, if need be by striking the first striking face 48 of the T-handle 40 or the second striking face 54 of the enlarged gripping portion 44, to free the blade from the bone. The foregoing process is repeated until a desired number of bone penetrations are achieved by the blade 16, such that sufficient bone is removed from contact with the implant to achieve removal.

Additionally, if implant removal with the starter blade of FIG. 9 is not sufficient then the finishing blade of FIG. 5 can be used to cut more/additional bone a deeper depth than the starter blade. The user inserts the finishing blade into a hole previously cut into the bone 300 by the starter blade. The user can then torque by hand or strike the strike plate 34 thereby driving the finishing blade further beneath the acetabular cup implant to be extracted. Indeed, the longitudinal dimension of the finishing blade is capable of extending to substantially a bottom portion of the acetabular cup implant to be extracted when fully inserted into the surrounding bone. The user then extracts the finishing blade, if need be by striking the first striking face 48 of the T-handle 40 or the second striking face 54 of the enlarged gripping portion 44 to free the blade from the bone. The user then inserts the finishing blade into another hole previously cut into the bone by the starter blade and strikes the strike plate 34 of the handle assembly to drive the finishing blade deep beneath the acetabular cup implant to be extracted. The finishing blade is then extracted as described and the process is repeated as necessary to remove the implant. The user can also exert a twisting force on the second handle 38 of the handle assembly 12 whereby the lateral cutting edges of the finishing blade cut bone that may be remaining between the puncture holes cut by the starter blade and/or the finishing blade to form a continuous substantially hemi-spherically shaped cut adjacent to the acetabular cup implant. Thereafter, the user may tilt the handle assembly 12 away from his or her body thereby causing the finishing blade to lift the acetabular cup implant from the surrounding cut bone.

Alternatively, the finishing blade may be used as the initial blade instead of or without the starter blade.

In accordance with the exemplary embodiments, there is provided an orthopedic instrument in the form of an acetabular cup extractor for reliably and efficiently extracting an acetabular cup implant from an acetabulum. For example, the handle assembly 12 has a strike plate 34 in-line with, i.e., directly above, the blade 16 resulting from the lateral offset of the longitudinal axes “A” and “B” of the second shaft portion 30 and the first shaft portion 28 of the first handle 26. Benefits realized by this structural arrangement include the tip of the blade 16 penetrating the bone surrounding the acetabular implant with optimum piercing and cutting force while ensuring that the guide 22 remains seated in the acetabular cup liner during cutting. Furthermore, a uniform cut is achieved by the curved blade thereby resulting in a minimum of bone being extracted from the acetabulum around the acetabular cup implant during the surgical procedure.

It will be appreciated by those skilled in the art that changes could be made to the exemplary embodiments described above without departing from the broad inventive concept thereof. It is to be understood, therefore, that this disclosure is not limited to the particular exemplary embodiments disclosed, but it is intended to cover modifications within the spirit and scope of the subject disclosure as defined by the appended claims.

I/We claim:

1. An acetabular cup extractor, comprising:
a handle assembly;
a quick connect coupling extending from the handle assembly;
a blade having:
a first quick connect for connecting with the quick connect coupling, and
a second quick connect; and
a guide configured to be received within an acetabular cup, the guide including a quick connect coupling for connecting with the second quick connect of the blade.

2. The acetabular cup extractor of claim 1, wherein the handle assembly comprises:
a first handle having:
a first shaft portion extending from the quick connect coupling; and
a second shaft portion extending from the first shaft portion and having a longitudinal axis laterally offset from a longitudinal axis of the first shaft portion.

3. The acetabular cup extractor of claim 2, wherein the second shaft portion is offset from the longitudinal axis of the first shaft portion a distance substantially equal to a distance a tip of the blade is offset from a longitudinal axis of the first quick connect.

4. The acetabular cup extractor of claim 2, wherein the blade has a tip substantially in-line with the longitudinal axis of the second shaft portion.

5. The acetabular cup extractor of claim 2, wherein the first handle further comprises a strike plate at its proximal end.

6. The acetabular cup extractor of claim 2, wherein the first handle further comprises a strike plate at its proximal end and positioned directly above the blade.

7. The acetabular cup extractor of claim 2, wherein the handle assembly further comprises:
a second handle having a longitudinal axis substantially transverse to a longitudinal axis of the first handle, the second handle having:
a T-handle at a first end of the second handle; and
an enlarged gripping portion about a second end of the second handle opposite the first end.

8. The acetabular cup extractor of claim 7, wherein the second handle includes a striking face.

9. The acetabular cup extractor of claim 7, wherein the T-handle includes a first striking face and a second striking face opposite the first striking face.

10. The acetabular cup extractor of claim 7, wherein the enlarged gripping portion includes a first striking face and a second striking face opposite the first striking face.

11. The acetabular cup extractor of claim 1, wherein the first quick connect extends coaxially with the second quick connect.

12. The acetabular cup extractor of claim 1, wherein the first quick connect extends from the blade in a first direction and the second quick connect extends from the blade in a second direction opposite the first direction.

13. The acetabular cup extractor of claim 1, wherein the blade includes a spear-point blade.

14. The acetabular cup extractor of claim 1, wherein the blade is curved in both its longitudinal direction and in a widthwise extent transverse to the longitudinal direction.

15. The acetabular cup extractor of claim 1, wherein the blade has an arc length of about 80 to 140 degrees.

16. The acetabular cup extractor of claim 1, wherein the guide is substantially shaped as a frustrum of a hemispherical dome.

17. The acetabular cup extractor of claim 1, wherein the guide includes a plurality of spaced apart scallop-shaped recesses circumscribing the guide.

18. An acetabular cup extractor, comprising:
a blade that includes:
a spear-point blade having a tip that is substantially in-line with the longitudinal axis of the second shaft portion, and wherein the blade is curved in both its longitudinal direction and in a widthwise extent transverse to the longitudinal direction,
a first quick connect, and
a second quick connect;
a quick connect coupling for connecting with the first quick connect of the blade;
a handle assembly that includes:
a first handle having:
a first shaft portion extending from the quick connect coupling,
a second shaft portion extending from the first shaft portion and having a longitudinal axis laterally offset from a longitudinal axis of the first shaft portion, and
a strike plate at its proximal end, and
a second handle having a longitudinal axis substantially transverse to a longitudinal axis of the first handle, the second handle having:
a T-handle at a first end of the second handle, and
an enlarged gripping portion about a second end of the second handle opposite the first end, wherein the enlarged gripping portion includes a planar striking face; and
a guide configured to be received within an acetabular cup, the guide including a quick connect coupling for connecting with the second quick connect of the blade.

19. The acetabular cup extractor of claim 18, wherein the guide is substantially shaped as a frustrum of a hemispherical dome.

20. The acetabular cup extractor of claim 18, wherein the guide includes a plurality of spaced apart scallop-shaped recesses circumscribing the guide.
